(19) United States
(12) Patent Application Publication
(10) Pub. No.: US 2006/0071096 A1
(43) Pub. Date: Apr. 6, 2006

(54) Control device for dispersing a liquid in a fluid

(76) Inventor: Antonin Vavra, Pooraka (AU)

Correspondence Address: F. Chau & Associates, 130 Woodbury Road, Woodbury, NY, 11797, US

(21) Appl. No.: 10/509,260

(22) PCT Filed: Mar. 26, 2003
(86) PCT No.: PCT/AU03/00375

§ 371 Date: Nov. 14, 2005

(30) Foreign Application Priority Data
Mar. 26, 2002 (AU) PS 1347
Feb. 4, 2003 (JP) 27244/2003

(51) Int. Cl. A61M 11/06

(52) U.S. Cl. 239/338

(57) ABSTRACT

The invention is directed to a control device for use with dispersion apparatus capable of dispersing a liquid in a fluid, such as a gas, to form a mist. The dispersion apparatus includes a chamber having a liquid-containing portion and a mist-containing portion. The control device includes a fluid inlet in communication with a fluid outlet, the fluid outlet being such that, in use, it is within the liquid-containing portion of the chamber. The control device also includes a mist outlet capable of being, in use, in communication with the mist-containing portion of the chamber. The fluid outlet includes means for controlling the flow of fluid therefrom such that, in use, either the degree of turbulence or the ratio of fluid to liquid in the mist can be controlled. The control device may also include a by-pass means between the fluid inlet and the mist outlet for assisting in controlling the ratio of liquid to fluid in the mist.

### FIELD OF THE INVENTION

The present invention relates to devices and apparatus for entraining or dispersing a liquid in a fluid. In most applications, the fluid will be a gas and the entraining or dispersing of the liquid therein will result in a mist being formed.

### BACKGROUND OF THE INVENTION

There are many fields of technology that utilize mists or dispersions for various methods or processes. Invariably, the mists or dispersions are desirable liquids entrained in a carrier gas that is either an active gas or is inert in the process or method. For example, it is known to entrain a spatter retardant in a gas used for arc welding. It is also known to apply liquid lubricants to various products by entraining the lubricant in oxygen or compressed air, as is similarly often done to apply anti-rust liquids to workpieces requiring rust-proofing.

With particular reference to arc welding, there have long been problems associated with the generation of small and minute pieces of metal, commonly called “spatter”, within and about the outlet end of a welding torch, and also about the weld-pool, the work-piece, and the weld itself. Conventionally, a welding operator is required to physically remove such spatter, which is often a physically demanding task sometimes requiring more than one operator, and which also introduces downtime.

Various attempts have been made to design heads and/or nozzles for welding torches that are capable of removing or reducing the spatter problem. Attempts have also been made to develop chemicals that may be applied directly to the weld torch and/or the weld-piece to prevent the spatter from attaching too rigidly thereto.

One attempt to reduce the spatter problem has been described in the applicant's own U.S. Pat. No. 5,603,854 where a system is described that disperses a liquid spatter retardant into the inert gas of an arc welding process. Whilst this system successfully reduces the effects of spatter (by reducing the ability of the spatter to adhere to the work-piece, the weld, the weld-pool, or the ancillary equipment), it does not readily allow for any degree of adjustment, as might be necessary when the system is used for a variety of different types of arc welding.

In this respect, some types of arc welding (such as hydrogen controlled welding) are often used to create welds that must be rated to withstand high pressures, such as in the fabrication of pressure vessels. These welds must meet stringent safety requirements in relation to the hydrogen content in the welds, in order to minimize the likelihood of cracks and/or fractures.

Therefore, for a particular welding activity where there are the dual aims of achieving a reduction in the effect of spatter yet also achieving low levels of hydrogen (indicated by low diffusible hydrogen levels in the weld), it is important that any spatter reduction system be able to be controlled so as to adjust various aspects of the dispersal of the spatter retardant.

With this in mind, as a part of the development process that lead to the present invention, the present inventor recognized that an increase in spatter retardant levels in the shielding gas of particular arc welding processes (which is desirable, in order to reduce the effects of spatter) appeared to result in undesirable increases in levels of diffusible hydrogen in the welds. The inventor thus recognized the need to be able to, in some situations, control the spatter retardant, in order to maximize the spatter retardant effects yet minimize diffusible hydrogen levels.

In turn, this lead to the development of a control device that can be used in any situation where it is desired to disperse or entrain a liquid in a fluid of some type. For instance, the same control aspects are equally desirable in a system used to disperse a lubricant or an anti-rust product into compressed air for a more controllable application.

An aim of the present invention is thus to provide a control device capable of being used in the dispersing of a liquid (such as a spatter retardant, a lubricant or a rust inhibiter) in a fluid (such as a shielding gas used for arc welding, or carrier gases used to deliver lubricants and rust inhibiters, in preferred forms). Following from that, the present invention also aims to provide a suitable dispersion apparatus that utilises the control device.

### SUMMARY OF THE INVENTION

The present invention provides a control device for use with dispersion apparatus capable of dispersing a liquid in a fluid to form a mist, the dispersion apparatus including a chamber having a liquid-containing portion and a mist-containing portion, the control device including:
a fluid inlet in communication with a fluid outlet, the fluid outlet being such that, in use, it is within the liquid-containing portion of the chamber; and
a mist outlet capable of being, in use, in communication with the mist-containing portion of the chamber;
   wherein the fluid outlet includes means for controlling the flow of fluid therefrom such that, in use, either the degree of turbulence or the ratio of fluid to liquid in the mist can be controlled.

In another form of the invention, there is provided a control device for use with dispersion apparatus capable of dispersing a liquid in a fluid to form a mist, the dispersion apparatus including a chamber having a liquid-containing portion and a mist-containing portion, the control device including:
a fluid inlet in fluid communication with a fluid outlet, the fluid outlet being such that, in use, it is within the retardant-containing portion of the chamber;
a mist outlet capable of being, in use, in fluid communication with the mist-containing portion of the chamber; and
the mist outlet being in fluid communication, via a by-pass means, with the fluid inlet;
   wherein the by-pass means allows control of the ratio of fluid to liquid in the mist.

The present invention also provides a dispersion apparatus that utilises the control device. The present invention thus provides an apparatus for dispersing a liquid in a fluid to form a mist, the apparatus including:
a chamber having a liquid-containing portion and a mist-containing portion;
a fluid inlet in fluid communication with a fluid outlet, the fluid outlet being within the liquid-containing portion of the chamber; and
a mist outlet in fluid communication with the mist-containing portion of the chamber;
   wherein the fluid outlet includes means for controlling the flow of gas therefrom such that, in use, either the degree of turbulence or the ratio of fluid to liquid in the mist can be controlled.

The present invention also provides an apparatus for dispersing a liquid in a gas to form a mist, the apparatus including:
a chamber having a liquid-containing portion and a mist-containing portion;
a fluid inlet in fluid communication with a fluid outlet, the fluid outlet being within the liquid-containing portion of the chamber;
a mist outlet in fluid communication with the mist-containing portion of the chamber, the mist outlet also being in fluid communication, via a by-pass means, with the fluid inlet;
   wherein the by-pass means allows control of the ratio of fluid to liquid in the mist.

The present invention further provides a control device for use with dispersion apparatus capable of dispersing a spatter retardant in a gas to form a mist, for use as a shielding gas for arc welding, the dispersion apparatus including a chamber having a spatter retardant containing portion and a mist-containing portion, the control device including:
a gas inlet in communication with a gas outlet, the gas outlet being such that, in use, it is within the spatter retardant containing portion of the chamber; and
a mist outlet capable of being, in use, in fluid communication with the mist-containing portion of the chamber;
   wherein the gas outlet includes means for controlling the flow of gas therefrom such that in use, either the degree of turbulence or the ration of gas to spatter retardant in the mist can be controlled.

The present invention also provides a control device for use with dispersion apparatus capable of dispersing a spatter retardant in a gas to form a mist for use as a shielding gas for arc welding; the dispersion apparatus including a chamber having a spatter retardant containing portion and a mist-containing portion, the control device including:
a gas inlet in communication with a gas outlet, the gas outlet being such that, in use, it is within the spatter retardant containing portion of the chamber;
a mist outlet capable of being, in use, in communication with the mist-containing portion of the chamber; and
the mist outlet being in communication, via a by-pass means, with the gas inlet;
   wherein the by-pass means allows control of the ratio of gas to spatter retardant in the mist.

The present invention also provides an apparatus for dispersing a spatter retardant in a gas to form a mist for use as a shielding gas for arc welding, the apparatus including:
a chamber having a spatter retardant containing portion and a mist-containing portion;
a gas inlet in communication with a gas outlet, the gas outlet being within the spatter retardant containing portion of the chamber; and
a mist outlet in communication with the mist-containing portion of the chamber;
   wherein the gas outlet includes means for controlling the flow of gas therefrom such that, in use, either the degree of turbulence or the ratio of gas to spatter retardant in the mist can be controlled.

The present invention also provides an apparatus for dispersing a spatter retardant in a gas to form a mist for use as a shielding gas for arc welding, the apparatus including:
a chamber having a spatter retardant containing portion and a mist-containing portion;
a gas inlet in communication with a gas outlet, the gas outlet being within the spatter retardant containing portion of the chamber; and
a mist outlet in communication with the mist-containing portion of the chamber;
   wherein the gas outlet includes means for controlling the flow of gas therefrom such that the ratio of gas to spatter retardant in the mist can be controlled.

Throughout this specification, the words ‘fluid’ and ‘mist’ will be used. For the sake of clarity, it should be understood that the word ‘fluid’ is being used to mean either a liquid or a gas, or indeed a mixture of a liquid and a gas. Having said that, it is envisaged that in most applications the fluid will be a gas. More often, the fluid will be a gas that is inert in relation to the liquid being dispersed and also in relation to the particular application.

Also, it should be understood that the word ‘mist’ is being used to generically describe a liquid dispersed or entrained in a fluid. No reference is being made to the particle size nor to the particles being such that they float in the gas (which they may or may not do), as might more often be understood by reference to a ‘mist’. In the majority of situations, where the fluid is a gas, this is a sensible description, and thus the description is being adopted throughout this specification for ease of reference. However, it is acknowledged that where the fluid itself is a liquid (or a liquid/gas mixture) this is not such a sensible description. Nonetheless, the specification in this case is acting as its own dictionary and is defining the manner in which the word is being used.

Finally, it should be appreciated that while this specification refers predominantly to the dispersing of a spatter retardant in a gas to form a mist for use as a shielding gas for arc welding, the invention is not to be limited only to that application. Also, a reference in this specification to a ‘spatter retardant’, a ‘retardant’, to ‘reduced spatter’, or to ‘reducing spatter’is a general reference to a fluid (normally a liquid) and its action in reducing the effects of spatter in an arc welding process. In an embodiment of the present invention, this reduction will normally be achieved by reducing the ability of the spatter to adhere to the work-piece, the tools or the weld itself, rather than by reducing the volume of spatter generated. Indeed, in some welding situations, the amount of spatter produced during the welding process may even increase, albeit in a form that will generally not adhere, as is required by the invention.

### GENERAL DESCRIPTION OF THE INVENTION

The dispersion apparatus with which the control device of the present invention is preferably used will ideally be provided by a canister or the like, which provides a chamber having the liquid-containing portion and the mist-containing portion, together with a suitable support means and suitable in-line connections. In this respect, suitable in-line connections will preferably be such as to allow the dispersion apparatus to be installed within the fluid line in an existing installation operation (such as an existing welding operation, lubricant supply system, or rust-inhibiter application process), and will usually cooperate with (or be integral with) the fluid inlet and the mist outlet described above as a part of the control device per se.

For ease of subsequent reference, further description of the present invention and its embodiments will be by way of the welding embodiment. Thus, the liquid will be a spatter retardant (noting the definitions provided above for that term) and the fluid will be an inert gas used as the shielding gas for an arc welding process.

In this embodiment, the canister thus acts as the source of the spatter retardant. In one form, the canister operates batch-wise, and may be refilled or replaced once the supply of spatter retardant is exhausted. In another form, the canister may be adapted to receive a continuous supply of spatter retardant, as necessary.

The canister may itself be an integral unit, unable to be opened without damaging the unit, or may be configured so as to be openable by the user or manufacturer. It may thus be provided as a lidded container, with the lid possibly being integral with, and thus forming a part of, the control device. Indeed, various elements of the control device may be advantageously provided as a part of a lid for the canister.

In one form of the invention, a filter may be provided between the retardant-containing portion and the mist-containing portion of the canister. Such a filter may simply be provided to prevent the generation of larger gaseous bubbles in the mist-containing portion, that may result in undesirable amounts of liquid moving through the mist outlet to the work-piece, as opposed to the controlled and reasonably finely dispersed liquid droplets that are preferentially formed in the mist. However, such a filter may itself also play a role in controlling the size and number of those liquid droplets.

Additionally (or alternatively), the filter may be provided between the mist-containing portion of the canister and the mist outlet, so as to provide an appropriate level of filtration to the mist before it exits the apparatus.

In relation generally to the mist-containing portion of the canister, it will of course be appreciated that, in use, the mist in the mist-containing portion is in fact transient, and merely passes through the mist-containing portion on its way to the mist outlet.

In a preferred form, the gas outlet of the control device incorporates the means for controlling the flow of gas. In its simplest form, the gas outlet is a nozzle that is capable of adjustment to permit differing flowrates and/or pressures of gas therefrom, and thus to allow control of the ratio of retardant to gas or the degree of turbulence generated upon mixing of the retardant and gas in the retardant-containing portion of the chamber. Preferably, the nozzle can be adjusted and set to provide a determined flowrate and/or pressure. In this way, the control device may be set by the manufacturer in advance of its use, or may be continuously adjusted by a user for each welding operation.

Preferably, the nozzle is provided at the free end of a probe that extends through the mist-containing portion of the chamber, through the filter (if present), and into the retardant-containing portion, such that the gas exits therefrom at or towards the bottom of the chamber.

In another preferred form of the present invention, there may be provided two paths for the flow of gas, one representing a total by-pass condition and the other representing a total mixing (of gas with retardant) condition. Preferably, the control device includes a by-pass means that permits the amount of gas by-passing the chamber to be controlled, thus providing a range of operational conditions between total by-pass and total mixing. The by-pass means may be adjustable continuously such that an operator is able to easily alter the adjustment in situ as necessary, and thus control the amount of gas by-passing he chamber. Alternatively, the adjustable by-pass means may be set at a predetermined position, requiring replacement, or a more rigorous in situ adjustment, to alter the adjustment and thus control the amount of gas by-passing the chamber.

Ideally, a triggering means is also provided to allow movement between the total by-pass condition and an operational condition, preferably such that the at rest position for the triggering means results in the operational condition being engaged. Thus, the triggering means must be manually manipulated in order to initiate the total by-pass condition. Of course, it will be appreciated that, in that total by-pass condition, all of the gas by-passes the chamber and thus passes through the control device without mixing with any of the spatter retardant.

It will be appreciated that, for the arc welding embodiment, the gas may be any suitable shielding gas, and may be an inert gas, an active gas, or a combination of inert gas and active gas. In particular, the shielding gas may be argon or an argon based mixture, carbon dioxide, oxygen, helium, hydrogen, or a mixture thereof. However, it will also be appreciated that in the broadest aspect of the present invention, the fluid (which is the shielding gas in the arc welding embodiment) may be any suitable fluid. For example, in the lubricant embodiment, the fluid will likely be an inert carrier gas such as compressed air, and similarly for the rust-inhibiting embodiment.

Further, for the arc welding embodiment, the spatter retardant may be any suitable spatter retardant, and may for instance be one or more suitable hydrocarbons, such as petroleum distillate. Again, as with the fluids mentioned above, the spatter retardant may of course be replaced with any suitable liquid that is required to be dispersed or entrained in the fluid. In the embodiments already described, the liquid would thus be a lubricant such as lubricating oils (typically the heavy distillates following kerosene in the fractional distillation of petroleum) or synthetic lubricants such as polyalphaolefins, polyglycols, polyol esters, diesteres and phosphate esters.

The liquid may also be any one of the large range of known rust inhibitors or a mixture of such known compounds.

Further still, the various applications for which the present invention will find use are numerous. For example, in the welding embodiment, the types of welding processes with which the present invention may find use are numerous, but will all be of the general type that is categorized as arc welding, namely fusion welding in which heat for welding is obtained from an electric arc or arcs.

For example, suitable types of arc welding would be manual and gravity metal arc welding, such as flux cored arc welding (FCAW), gas metal arc welding (GMAW), pulsed arc welding (PAW) and resistance welding. Examples of the more common gas metal arc welding (GMAW) are CO₂ welding, metal inert gas arc welding (MIG) and metal active gas arc welding (MAG). All of these welding techniques may be conducted either using robotic or hand operated welding equipment.

### BRIEF DESCRIPTION OF THE DRAWINGS

An embodiment of the invention will now be described, by way of example only, with reference to the accompanying drawings, in which:

FIG. 1 is a perspective view of a dispersion apparatus in accordance with an embodiment of the invention related to the dispersion of a spatter retardant in a shielding gas for use in arc welding;

FIG. 2 is a cut away half view of the apparatus shown in FIG. 1; and

FIG. 3 is a mid-line cross-sectional view of the apparatus shown in FIG. 1.

### DETAILED DESCRIPTION OF THE PREFERRED EMBODIMENT

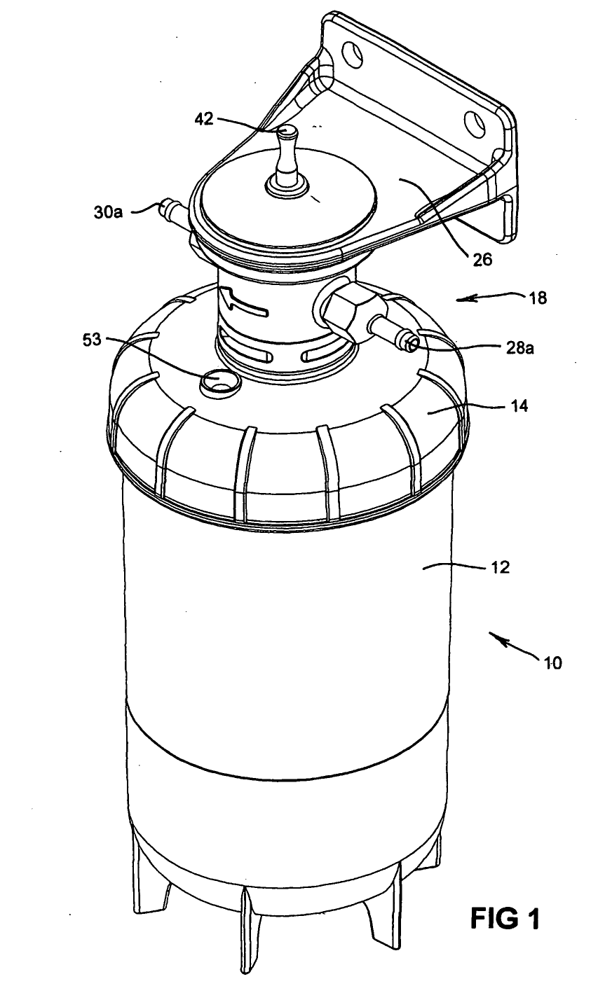

FIG. 1 illustrates a dispersion apparatus 10 capable of dispersing a liquid (such as a spatter retardant) in a fluid (such as a gas) to form a mist for use as a shielding gas when arc welding. The apparatus 10 includes a canister 12 that has a lid 14 threadedly engaged thereto. In this form of the invention, the canister is likely to be operated as a pressure vessel and thus includes a suitable pressure relief valve 53.

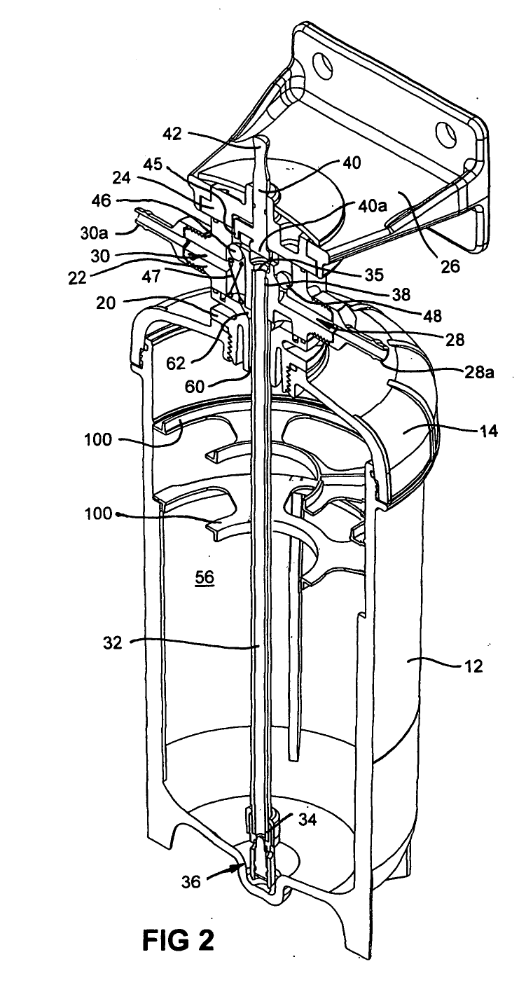

As shown in FIG. 2, mounted within a central aperture 16 of the lid 14 is a headpiece 18. The headpiece 18 includes a base 20, a middle body 22 and a top 24. Attached to the top 24 is a support bracket 26 by which the apparatus 10 can be mounted to a support structure (not shown).

The middle body 22 includes a gas inlet 28 and a mist outlet 30. A barb 28a, 30a is connected respectively to the gas inlet 28 and the mist outlet 30 to enable connection to respective flexible pipes.

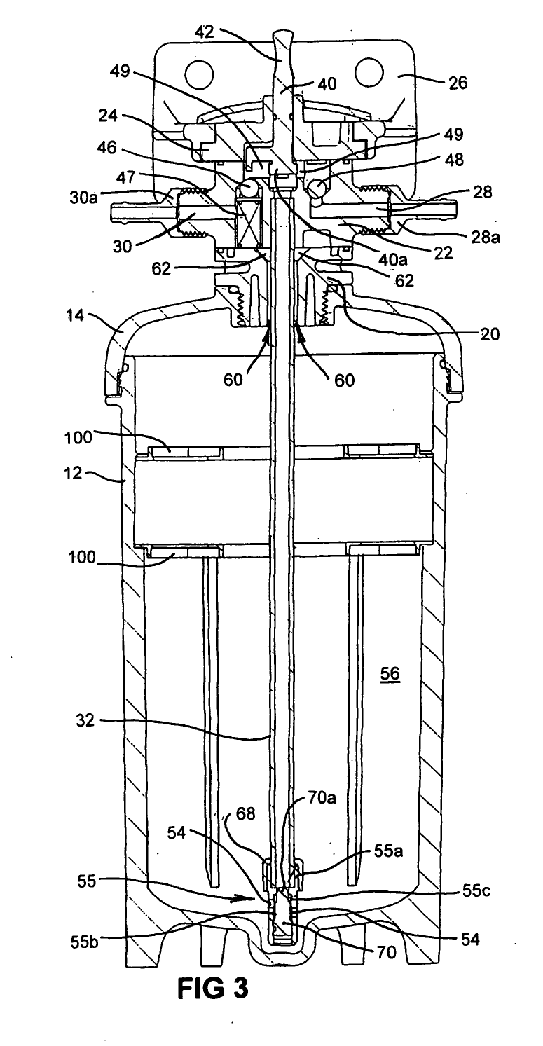

The middle body 22 also includes a centrally located probe 32. As shown in FIGS. 2 and 3, the probe 32 is integral with the middle body 22 and extends into the canister 12. The probe 32 has a lower end 34 that includes a gas outlet incorporated with a gas flow controlling means, which together form a flow control device 36. The upper end 38 of the probe 32 opens into a bore 35. Positioned above the bore 35, and arranged for up and down movement within the headpiece 18, is a plunger 40. The plunger 40 has a top 42 that protrudes from the lid 14. The plunger 40 and its top 42 provide the device with a triggering means for moving between the total by-pass condition and an operational condition described further below.

In this embodiment, the control device of the present invention includes the gas inlet 28, the mist outlet 30, and the gas outlet that generally includes the lower end 34 of the probe 32 with its flow control device 36. This is probably best envisaged as being that part of the apparatus that would remain if the canister 12, its lid 14, and the support bracket 26 were removed.

Returning to a more detailed description of the dispersion apparatus as a whole, the middle body 22 (and thus, in effect, the control device of the invention) is configured so that there are two different fluid paths for gas to flow therethrough, a first flow path that represents a total by-pass condition (and thus is referred to as a total by-pass path) and a second flow path that represents an operational condition (and thus is referred to as an operational path).

The total by-pass path is configured so that gas entering the gas inlet 28 flows directly towards the mist outlet 30 without passing into the probe 32. The total by-pass path is triggered via the top 42 of the plunger 40 being depressed so as to close off the upper end 38 of the probe 32 to the flow of gas.

In this respect, depression of the top 42 of the plunger 40 causes a plug portion 40a of the plunger 40 to move into the bore 35 and thereby close off the bore 35. Depression of the plunger 40 also causes an arm 45 that extends from the plunger 40 to press against, and thereby open, the spring biased ball valve 46. This enables gas to pass through the gas inlet 28, into contact with a ball valve 48 so as to open the valve 48, through a pathway 49 which extends behind the plug portion 40a of the plunger 40, through the spring biased ball valve 46 and out through the mist outlet 30.

The operational path through the middle body 22 is one that enables the gas to pass through the gas inlet 28, through the bore 35 and into the upper end 38 of the probe 32. The operational path will be engaged when the plunger 40 is not depressed.

When the operational path is engaged, gas enters the gas inlet 28, presses against the ball valve 48 to open the valve, passes through the bore 35, into the upper end 38 of the probe 32 and then flows down along the probe 32 towards the lower end 34 thereof. The gas then passes through the flow control device 36, out through the apertures 54 formed in a housing 55 of the flow control device 36 and into the chamber 56 defined by the canister 12 and the lid 14.

However, in a preferred form, the control device of the invention includes a by-pass means in the form of the spring 47 of the spring biased ball valve 46 mentioned above. Ideally, the spring 47 (which may of course be any type of resilient member that will achieve the same purpose as the spring) is selected to have a predetermined resilience that permits gas flow at a certain pressure to open the ball valve 46 and permit passage of a certain amount of gas therepast.

In this way, an operational condition may be created where a predetermined amount of gas is allowed to by-pass the chamber 56, and then subsequently mix with the mist exiting the mist outlet 30. This provides additional control to the ratio of the retardant to gas in the mist exiting the mist outlet 30. Of course, it will be appreciated that other operational conditions may thus be created, simply by utilizing a spring (or other such resilient member) having a different resilience and thus permitting passage of different amounts of gas at the same gas pressure. Indeed, a range of control devices may thus be manufactured that each provide a predetermined by-pass ratio and thus a predetermined ratio of retardant to gas in the mist exiting the mist outlet 30.

Alternatively, a resilient member may be utilized that includes an adjustment means that permits adjustment of the resilience thereof in situ. This would negate the need to provide a range of differing control devices, and would allow an operator to select a suitable ratio of retardant to gas during operation.

In use, the chamber 56 includes a retardant-containing portion (i.e. the lower portion of the chamber) and a mist-containing portion (i.e. the upper portion of the chamber), and may of course include a pressure relief valve (not shown) as necessary. The lower portion of the chamber 56 contains, in use, a spatter retardant. When gas passes through the apertures 54 of the flow control device 36, the gas is dispersed through the spatter retardant. This results in a mist forming in the mist-containing portion (i.e. the upper portion) of the chamber 56.

The mist, which is a mixture of the gas and the spatter retardant (the spatter retardant being entrained in liquid droplets in the gas), flows through the mist channels 60, which are formed in the base 20 of the headpiece 18. Each mist channel 60 has an upper opening 62 that is in fluid communication with the mist outlet 30. The spring biased ball valve 46 prevents the mist from back flowing through the pathway 49, although there may be a higher pressure amount of unmixed gas flowing through past the ball valve 46 if the by-pass means embodiment of the invention is being utilized.

Thus, it will be appreciated that when the apparatus 10 is in use and the plunger 40 is not depressed, the shielding gas exiting the mist outlet 30 will contain an amount of spatter retardant. As will be explained in more detail subsequently, the amount of spatter retardant contained in the shielding gas exiting the mist outlet 30 will depend on either the configuration of the flow control device 36 or the operation of the by-pass means, or both.

Mounted within the mist-containing portion of the chamber 56 is a pair of filter supports 100. Each filter support 100 is arranged to be able to seat a filter pad (not shown). The filter pads are arranged to filter the mist before it passes through the mist outlets 60. Preferably, the filter pads are present in order to prevent gas bubbles from forming in the mist-containing portion of the chamber 56. However, the filter pads may also be selected such that they impact on the size of the liquid droplets in the mist. Of course, filter pads need not be used at all.

In the illustrated embodiment of the present invention, the flow control device 36 includes the housing 55, a nut 68 and a control pin 70. The nut 68 is secured to the lower end 34 of the probe 32 in a manner whereby the housing 55 can be threadedly secured to the nut 68. The housing 55 includes an upper bore 55a and a lower bore 55b. The upper and lower bores 55a , 55b are joined by a middle bore 55c which has a reduced diameter. When the housing 55 is secured to the nut 68, the lower end 34 of the probe 32 is located in the upper bore 55a. The control pin 70 is mounted in the lower bore 55b of the housing 55 so that a top end 70a can be arranged to extend into the middle bore 55c, and may extend into the upper bore 55a.

The control pin 70 is mounted within the housing 55 so that rotation of the control pin 70 in one direction drives the control pin 70 upwardly towards the upper bore 55a. This restricts the flow of gas into the lower bore 55b and consequently the flow of gas out through the apertures 54. Rotation of the control pin 70 in the opposite direction withdraws the control pin 70 away from the upper bore 55a so as to enable an increased flow of gas through the apertures 54.

By varying the position of the control pin 70 within the housing 55, the amount of gas passing into the lower bore 55b and through the apertures 54 can be altered so as to ultimately control the composition of the mist which is formed in the upper portion of the chamber 56.

The top end 70a of the control pin 70 may be shaped to vary the nature of the flow of the gas as it passes over the control pin 70 and flows into the lower bore 55b and out through the apertures 54. The amount of turbulence in the flow may thus also be varied, in this embodiment by virtue of the shape of the control pin 70, so as to vary the amount of spatter retardant in the resulting mist.

Having described one form of flow control device 36, it will be appreciated by those skilled in the art that the flow control device 36 may adopt different configurations.

Finally, the embodiment has been described by way of example only and modifications within the spirit and scope of the invention are anticipated.

1. A control device for use with dispersion apparatus capable of dispersing a liquid in a fluid to form a mist, the dispersion apparatus including a chamber having a liquid-containing portion and a mist-containing portion, the control device including:
a fluid inlet in communication with a fluid outlet, the fluid outlet being such that, in use, it is within the liquid-containing portion of the chamber; and
a mist outlet capable of being, in use, in fluid communication with the mist-containing portion of the chamber;
wherein the fluid outlet includes means for controlling the flow of fluid therefrom such that, in use, either the degree of turbulence or the ratio of fluid to liquid in the mist can be controlled.

2. A control device according to claim 1, wherein the fluid outlet incorporates the means for controlling the flow of fluid.

3. A control device according to claim 2, wherein the fluid outlet is a nozzle that is capable of adjustment to provide a predetermined flowrate and/or pressure of fluid to control the degree of turbulence of the fluid exiting the nozzle or the ratio of fluid to liquid in the mist.

4. A control device according to claim 3, wherein the nozzle is provided at the free end of a probe configured so as to, in use, extend through the mist-containing portion of the chamber into the liquid-containing portion, such that the fluid exits therefrom at or towards the bottom of the chamber.

5. A control device according to any one of claims 1 to 4, including two paths for the flow of fluid, one representing a total by-pass condition and the other representing a total mixing condition.

6. A control device according to claim 5, including a by-pass means that permits the amount of fluid by-passing the chamber to be controlled, thus providing a range of operational conditions between total by-pass and total mixing.

7. A control device according to claim 6, wherein the by-pass means is adjustable continuously such that an operator is able to easily alter in situ the amount of fluid by-passing the chamber.

8. A control device according to claim 6, wherein the by-pass means may be set at a predetermined position, requiring replacement to alter the amount of fluid by-passing the chamber.

9. A control device according to any one of claims 5 to 8, wherein a triggering means is provided to move between the total by-pass condition and an operational condition, such that the at-rest position for the triggering means results in the operational condition being engaged.

10. A control device according to claim 9, wherein the triggering means, when moved away from its at-rest position, in the total by-pass condition prevents the passage of fluid to the fluid outlet but permits the passage of fluid to the mist outlet.

11. A control device according to claim 9 or claim 10, wherein the triggering means is a plunger capable of external manual manipulation.

12. A control device for use with dispersion apparatus capable of dispersing a liquid in a fluid to form a mist, the dispersion apparatus including a chamber having a liquid-containing portion and a mist-containing portion, the control device including:
a fluid inlet in communication with a fluid outlet, the fluid outlet being such that, in use, it is within the liquid-containing portion of the chamber; and
a mist outlet capable of being, in use, in communication with the mist-containing portion of the chamber;
wherein the fluid outlet includes means for controlling the flow of fluid therefrom such that, in use, the ratio of fluid to liquid in the mist can be controlled.

13. A control device for use with dispersion apparatus capable of dispersing a liquid in a fluid to form a mist, the dispersion apparatus including a chamber having a liquid-containing portion and a mist-containing portion, the control device including:
a fluid inlet in communication with a fluid outlet, the fluid outlet being such that, in use, it is within the liquid-containing portion of the chamber;
a mist outlet capable of being, in use, in communication with the mist-containing portion of the chamber; and
the mist outlet being in communication, via a by-pass means, with the fluid inlet;
wherein the by-pass means allows control of the ratio of fluid to liquid in the mist

14. An apparatus for dispersing a liquid in a fluid to form a mist, the apparatus including:
a chamber having a liquid-containing portion and a mist-containing portion;
a fluid inlet in communication with a fluid outlet, the fluid outlet being within the liquid-containing portion of the chamber; and
a mist outlet in communication with the mist-containing portion of the chamber;
wherein the fluid outlet includes means for controlling the flow of fluid therefrom such that, in use, either the degree of turbulence or the ratio of fluid to liquid in the mist can be controlled.

15. An apparatus according to claim 14, wherein the chamber is provided by a canister.

16. An apparatus according to claim 14 or claim 15, wherein a filter is provided between the liquid-containing portion and the mist-containing portion.

17. An apparatus according to any one of claims 14 to 16 wherein the fluid outlet incorporates the means for controlling the flow of fluid.

18. An apparatus according to claim 17, wherein the fluid outlet is a nozzle that is capable of adjustment to provide a predetermined flowrate and/or pressure of fluid to control the degree of turbulence of the fluid exiting the nozzle or the ratio of fluid to liquid in the mist.

19. An apparatus according to claim 18 wherein the nozzle is provided at the free end of a probe configured so as to, in use, extend through the mist-containing portion of the chamber into the liquid-containing portion, such that the fluid exits therefrom at or towards the bottom of the chamber.

20. An apparatus according to any one of claims 14 to 19, including two paths for the flow of fluid, one representing a total by-pass condition and the other representing a total mixing condition.

21. An apparatus according to claim 20, including a by-pass means that permits the amount of fluid by-passing the chamber to be controlled, thus providing a range of operational conditions between total by-pass and total mixing.

22. An apparatus according to claim 21, wherein the adjustable by-pass means is adjustable continuously such that an operator is able to in situ alter the amount of fluid by-passing the chamber.

23. An apparatus according to claim 21, wherein the adjustable by-pass means may be set at a predetermined position, requiring replacement to alter the amount of fluid by-passing the chamber.

24. An apparatus according to any one of claims 20 to 23, wherein a triggering means is provided to move between the total by-pass condition and an operational condition, such that the at-rest position for the triggering means results in the operational condition being engaged.

25. An apparatus according to claim 24, wherein the triggering means, when moved away from its at-rest position, in the total by-pass condition prevents the passage of fluid to the fluid outlet but permits the passage of fluid to the mist outlet.

26. An apparatus according to claim 24 or claim 25, wherein the triggering means is a plunger capable of external manual manipulation.

27. An apparatus for dispersing a liquid in a fluid to form a mist, the apparatus including:
a chamber having a liquid-containing portion and a mist-containing portion;
a fluid inlet in communication with a fluid outlet, the fluid outlet being within the liquid-containing portion of the chamber; and
a mist outlet in communication with the mist-containing portion of the chamber;
wherein the fluid outlet includes means for controlling the flow of fluid therefrom such that the ratio of fluid to liquid in the mist can be controlled.

28. An apparatus for dispersing a liquid in a fluid to form a mist, the apparatus including:
a chamber having a liquid-containing portion and a mist-containing portion;
a fluid inlet in communication with a fluid outlet, the fluid outlet being within the liquid-containing portion of the chamber;
a mist outlet in communication with the mist-containing portion of the chamber, the mist outlet also being in communication, via a by-pass means, with the fluid inlet;
wherein the by-pass means allows control of the ratio of fluid to liquid in the mist.

29. A control device for use with dispersion apparatus capable of dispersing a spatter retardant in a gas to form a mist for use as a shielding gas for arc welding, the dispersion apparatus including a chamber having a spatter retardant containing portion and a mist-containing portion, the control device including:
a gas inlet in communication with a gas outlet, the gas outlet being such that, in use, it is within the spatter retardant containing portion of the chamber; and
a mist outlet capable of being, in use, in fluid communication with the mist-containing portion of the chamber;
wherein the gas outlet includes means for controlling the flow of gas therefrom such that, in use, either the degree of turbulence or the ratio of gas to spatter retardant in the mist can be controlled.

30. A control device according to claim 29, wherein the gas outlet incorporates the means for controlling the flow of gas.

31. A control device according to claim 30, wherein the gas outlet is a nozzle that is capable of adjustment to provide a predetermined flowrate and/or pressure of gas to control the degree of turbulence of the gas exiting the nozzle or the ratio of gas to spatter retardant in the mist.

32. A control device according to claim 31, wherein the nozzle is provided at the free end of a probe configured so as to, in use, extend through the mist-containing portion of the chamber into the spatter retardant containing portion, such that the gas exits therefrom at or towards the bottom of the chamber.

33. A control device according to any one of claims 29 to 32, including two paths for the flow of gas, one representing a total by-pass condition and the other representing a total mixing condition.

34. A control device according to claim 33, including a by-pass means that permits the amount of gas by-passing the chamber to be controlled, thus providing a range of operational conditions between total by-pass and total mixing.

35. A control device according to claim 34, wherein the by-pass means is adjustable continuously such that an operator is able to easily alter in situ the amount of gas by-passing the chamber.

36. A control device according to claim 34, wherein the by-pass means may be set at a predetermined position, requiring replacement to alter the amount of gas by-passing the chamber.

37. A control device according to any one of claims 33 to 36, wherein a triggering means is provided to move between the total by-pass condition and an operational condition, such that the at-rest position for the triggering means results in the operational condition being engaged.

38. A control device according to claim 37, wherein the triggering means, when moved away from its at-rest position, in the total by-pass condition prevents the passage of gas to the gas outlet but permits the passage of gas to the mist outlet.

39. A control device according to claim 37 or claim 38, wherein the triggering means is a plunger capable of external manual manipulation.

40. A control device for use with dispersion apparatus capable of dispersing a spatter retardant in a gas to form a mist for use as a shielding gas for arc welding, the dispersion apparatus including a chamber having a spatter retardant containing portion and a mist-containing portion, the control device including:
a gas inlet in communication with a gas outlet, the fluid outlet being such that, in use, it is within the spatter retardant containing portion of the chamber; and
a mist outlet capable of being, in use, in communication with the mist-containing portion of the chamber;
wherein the gas outlet includes means for controlling the flow of gas therefrom such that, in use, the ratio of gas to spatter retardant in the mist can be controlled.

41. A control device for use with dispersion apparatus capable of dispersing a spatter retardant in a gas to form a mist for use as a shielding gas for arc welding, the dispersion apparatus including a chamber having a spatter retardant containing portion and a mist-containing portion, the control device including:
a gas inlet in communication with a gas outlet, the gas outlet being such that, in use, it is within the spatter retardant containing portion of the chamber;
a mist outlet capable of being, in use, in communication with the mist-containing portion of the chamber; and
the mist outlet being in communication, via a by-pass means, with the gas inlet;
wherein the by-pass means allows control of the ratio of gas to spatter retardant in the mist.

42. An apparatus for dispersing a spatter retardant in a gas to form a mist for use as a shielding gas for arc welding, the apparatus including:
a chamber having a spatter retardant containing portion and a mist-containing portion;
a gas inlet in communication with a gas outlet, the gas outlet being within the spatter retardant containing portion of the chamber; and
a mist outlet in communication with the mist-containing portion of the chamber;
wherein the gas outlet includes means for controlling the flow of gas therefrom such that, in use, either the degree of turbulence or the ratio of gas to spatter retardant in the mist can be controlled.

43. An apparatus according to claim 42, wherein the chamber is provided by a canister.

44. An apparatus according to claim 42 or claim 43, wherein a filter is provided between the spatter retardant containing portion and the mist-containing portion.

45. An apparatus according to any one of claims 42 to 44 wherein the gas outlet incorporates the means for controlling the flow of gas.

46. An apparatus according to claim 45, wherein the gas outlet is a nozzle that is capable of adjustment to provide a predetermined flowrate and/or pressure of gas to control the degree of turbulence of the gas exiting the nozzle or the ratio of gas to spatter retardant in the mist.

47. An apparatus according to claim 46 wherein the nozzle is provided at the free end of a probe configured so as to, in use, extend through the mist-containing portion of the chamber into the spatter retardant containing portion, such that the gas exits therefrom at or towards the bottom of the chamber.

48. An apparatus according to any one of claims 42 to 47, including two paths for the flow of gas, one representing a total by-pass condition and the other representing a total mixing condition.

49. An apparatus according to claim 48, including a by-pass means that permits the amount of gas by-passing the chamber to be controlled, thus providing a range of operational conditions between total by-pass and total mixing.

50. An apparatus according to claim 49, wherein the adjustable by-pass means is adjustable continuously such that an operator is able to in situ alter the amount of gas by-passing the chamber.

51. An apparatus according to claim 49, wherein the adjustable by-pass means may be set at a predetermined position, requiring replacement to alter the amount of gas by-passing the chamber.

52. An apparatus according to any one of claims 48 to 51, wherein a triggering means is provided to move between the total by-pass condition and an operational condition, such that the at-rest position for the triggering means results in the operational condition being engaged.

53. An apparatus according to claim 52, wherein the triggering means, when moved away from its at-rest position, in the total by-pass condition prevents the passage of gas to the gas outlet but permits the passage of gas to the mist outlet.

54. An apparatus according to claim 52 or claim 53, wherein the triggering means is a plunger capable of external manual manipulation.

55. An apparatus for dispersing a spatter retardant in a gas to form a mist for use as a shielding gas for arc welding, the apparatus including:
a chamber having a spatter retardant containing portion and a mist-containing portion;
a gas inlet in communication with a gas outlet, the gas outlet being within the spatter retardant containing portion of the chamber; and
a mist outlet in communication with the mist-containing portion of the chamber;
wherein the gas outlet includes means for controlling the flow of gas therefrom such that the ratio of gas to spatter retardant in the mist can be controlled.

56. An apparatus for dispersing a spatter retardant in a gas to form a mist for use as a shielding for arc welding, the apparatus including:
a chamber having a spatter retardant containing portion and a mist-containing portion;
a gas inlet in communication with a gas outlet, the gas outlet being within the spatter retardant containing portion of the chamber;
a mist outlet in communication with the mist-containing portion of the chamber, the mist outlet also being in communication, via a by-pass means, with the gas inlet;
wherein the by-pass means allows control of the ratio of gas to spatter retardant in the mist.

57. A control device according to claim 1, 12, 13, 29, 40 or 41 substantially as herein described in relation to the accompanying drawings.

58. A dispersion apparatus according to claim 14, 27, 28 42, 55 or 56 substantially as herein described in relation to the accompanying drawings.
